(19) United States
(12) Patent Application Publication
(10) Pub. No.: US 2002/0187490 A1
(43) Pub. Date: Dec. 12, 2002

(54) Microbial identification chip based on DNA-DNA hybridization

(75) Inventors: James Tiedje, Lansing, MI (US); Jae-Chang Cho, East Lansing, MI (US)

Correspondence Address: MEDLEN & CARROLL, LLP, Suite 350, 101 Howard Street, San Francisco, CA, 94105, US

(73) Assignee: Michigan State University, East Lansing, MI (US)

(21) Appl. No.: 10/073,464

(22) Filed: Feb. 11, 2002

Related U.S. Application Data
(60) Provisional application No. 60/296,982, filed on Jun. 7, 2001.

(51) Int. Cl. C12Q 1/68; C12M 1/34

(52) U.S. Cl. 435/6; 435/287.2

(57) ABSTRACT

The present invention provides methods and compositions for the identification of microbial species from a variety of sources, including clinical specimens, food, environmental samples, waste or drinking water samples and industrial samples. In particularly preferred embodiments, the present invention provides a DNA “chip” containing bacterial genomic sequences arranged in a microarray for bacterial identification.

The present application claims priority benefit to U.S. Provisional Patent Application No. 60/296,982, filed Jun. 7, 2001.

This invention was made with government support from the National Science Foundation, grant numbers DEB-0075564 and DEB-9120006. The United States Government has certain rights in the invention.

### FIELD OF THE INVENTION

The present invention provides methods and compositions for the identification of microbial species from a variety of sources, including clinical specimens, food, environmental samples, waste or drinking water samples and industrial samples. In particularly preferred embodiments, the present invention provides a DNA “chip” containing bacterial genomic sequences arranged in a microarray for bacterial identification.

### BACKGROUND OF THE INVENTION

Bacterial identification methods currently used include analysis of morphological, physiological, biochemical, and genetic data. In the last two decades, molecular methods, especially 16S rRNA gene sequencing, have developed into reliable aids for the identification of diverse bacteria. Although the 16S rRNA method has served as a powerful tool for finding phylogenetic relationship between bacteria because of its molecular clock properties and the large database for sequence comparison, the molecule is too conserved to provide good resolution at the species and subspecies levels (See e.g., Woese, Microbiol. Rev., 51:221-271 [1987]; DeParasis and Roth, Phytopathol., 80:618-621 [1990]; Fox et al., Int. J. Syst. Bacteriol., 42:166-170; Martinez-Murcia et al., Int. J. Syst. Bacteriol., 42:412-421 [1992]; Stackebrandt and Goebel, Int. J. Syst. Bacteriol., 44:846-849 [1994] and Weisburg et al., J. Bacteriol., 173:697-703 [1991]).

The relationship between 16S rRNA gene similarity and percent DNA-DNA reassociation is a logarithmic function in which the sequence similarity within a species (>70% DNA relatedness) is expected to be >98% (Devereux et al., J. Bacteriol., 172:3609-3619 [1990]), and the similarity among different species in a genus (e.g., fluorescent Pseudomonas) is 93.3 to 99.9% (Moore et al., Syst. Appl. Microbiol., 19:478-492 [1996]). Considering the high sequence conservation and relative standard errors at 98% and 90% sequence similarities of 19% and 8%, respectively (Keswani et al., Int. J. Syst. Bacteriol., 46:727-735 [1996]), 16S rDNA analysis results on closely related strains may be inaccurate and inconsistent with the results obtained by other methods. Incongruity between genome structure and 16S rDNA sequence similarity has also been reported (See, Lessie et al., FEMS Microbiol. Lett., 144:117-128 [1996]). As many important ecological and clinical characteristics of bacteria such as pathogenicity, competitiveness, substrate range, and bioactive molecule production, tend to vary below the species level, methods with higher resolution than 16S rDNA sequence are needed.

DNA-DNA hybridization methods provide more resolution than 16S rDNA sequencing, and the 70% criterion (Wayne et al., Int. J. Syst. Bacteriol., 37:463-464 [1987]) has been a cornerstone for describing a bacterial species. Nonetheless, these methods are not popular, largely due to major disadvantages such as the laborious nature of pairwise cross-hybridizations, the requirement for isotope use, and the fact that it is impossible to establish a central database using these methods. Thus, there remains a need for easy-to-use methods to identify and type microorganisms based on DNA-DNA homologies that eliminate these disadvantages.

### SUMMARY OF THE INVENTION

The present invention provides methods and compositions for the identification of microbial species from a variety of sources, including clinical specimens, food, environmental samples, waste or drinking water samples and industrial samples. In particularly preferred embodiments, the present invention provides a DNA “chip” containing bacterial genomic sequences arranged in a microarray for bacterial identification.

In some embodiments, the present invention provides methods for identifying bacteria, comprising providing genomic sequences from a plurality of bacterial species arrayed on a solid support so as to create a plurality of arrayed elements, and labeled target DNA from a test bacteria of interest, and labeled reference DNA from the strains of bacteria represented on the solid support; hybridizing the target and reference DNA to the arrayed sequences to produce a hybridization pattern, wherein each hybridized DNA in the hybridization pattern has a signal; and calculating the ratio of each hybridization signal intensity at each array element to determine the identity of the test bacteria. In some embodiments, the test bacteria are from a sample obtained from a subject. In alternative embodiments, the test bacteria are pathogenic organisms. In still further embodiments, the test bacteria are clinical samples, while in other embodiments, the test bacteria are environmental isolates. In still further embodiments, the test bacteria are veterinary, food, or other isolates (e.g., feed, water, etc.). In yet other embodiments, the reference bacteria are clinical, environmental, veterinary, food, or other isolates (e.g., feed, water, etc.). In some particularly preferred embodiments, the solid support is a microchip. In further embodiments, the calculating comprises statistical analysis. In still further embodiments, the methods further comprise the step of producing hybridization profiles of the test and reference bacteria. In some preferred embodiments, the signal comprises fluorescence.

The present invention also provides methods for identifying bacteria, comprising: providing genomic sequences from a plurality of bacterial species arrayed on at least one microchip, so as to create a plurality of arrayed elements, and labeled target DNA from a test bacteria of interest, and labeled reference DNA from the strains of bacteria represented on the microchip(s); hybridizing the target and reference DNA to the arrayed sequences to produce a hybridization pattern, wherein each hybridized DNA in the hybridization pattern has a signal; and calculating the ratio of each hybridization signal intensity at each array element to determine the identity of the test bacteria. In some embodiments, the test bacteria are from a sample obtained from a subject. In alternative embodiments, the test bacteria are pathogenic organisms. In still further embodiments, the test bacteria are clinical samples, while in other embodiments, the test bacteria are environmental isolates. In still further embodiments, the test bacteria are veterinary, food, or other isolates (e.g., feed, water, etc.). In yet other embodiments, the reference bacteria are clinical, environmental, veterinary, food, or other isolates (e.g., feed, water, etc.). In further embodiments, the calculating comprises statistical analysis. In still further embodiments, the methods further comprise the step of producing hybridization profiles of the test and reference bacteria. In some preferred embodiments, the signal comprises fluorescence.

The present invention also provides kits for identification of bacteria, comprising genomic sequences from a plurality of bacterial species arrayed on a solid support so as to create a plurality of arrayed elements, and labeled reference DNA from the strains of bacteria represented on the solid support. In one preferred embodiment, the solid support comprises at least one microchip. In another preferred embodiment, the labeled reference DNA is labeled with a fluorescent label. In additional embodiments, the reference DNA is obtained from organisms selected from the group consisting of pathogenic bacteria and environmental bacteria. In still further preferred embodiments, the genomic sequences arrayed on the solid support are labeled. In yet other embodiments, the genomic sequences arrayed on the solid support are labeled with a fluorescent label.

In still further embodiments, the array hybridization profiles (i.e., signal ratios) and/or raw images (e.g., microarray scans) are archived in a web server to establish a central database. This central database finds use by researchers who compare their results with those in the database in order to identify their strains in a manner that is analogous to retrieval of RDP data.

### DESCRIPTION OF THE FIGURES

FIG. 1 provides a scatter plot diagram of hybridization profiles of P. fluorescens ATCC 13525^{T}. Results from triplicate hybridization experiments (r²=0.94) are displayed. Each axis (x, y, and z) represents the log-transformed hybridization signal ratios from each experiment.

FIG. 2 shows the relationship between previously reported whole genomic DNA homology values and similarity values obtained by one embodiment of the method of the present invention. The solid line indicates the regression curve, while the dotted line indicates the 95% prediction interval, respectively.

FIG. 3 provides a similarity dendrogram (UPGMA) of hybridization profiles of fluorescent Pseudomonas strains, created using one embodiment of the present invention. The solid line indicates a cut-off value at which all of the different strains tested were resolved. The dashed line indicates species level resolution that corresponds to 70% whole genomic DNA hybridization.

FIG. 4 provides a similarity dendrogram (UPGMA) of hybridization profiles of 338 genome fragments spotted on the microarray of one embodiment of the present invention. Cluster F (98.7%), C (94.1%), A (91.8%), P (100%), and Y (100%) are comprised of genome fragments from the reference strains P. fluorescens (ATCC 13525^{T}), P. chlororaphis (ATCC 9447), P. aeruginosa (ATCC 15692), and P. putida (ATCC 12633^{T}). Clusters V to Z comprised genome fragments from different reference strains, except cluster Y.

FIG. 5, Panel A provides an evenness value (θ_{E}) scatter diagram, with the average and standard deviation of log hybridization signal ratio indicated.

FIG. 5, Panel B provides θ_{E} values by genome fragment, ID 1-92, 93-182, 183-278, and 279-338, which originated from P. fluorescens (ATCC 13525^{T}), P. chlororaphis (ATCC 9447), P. putida (ATCC 12633^{T}), and P. aeruginosa (ATCC 15692), respectively. In this Figure, the solid line indicates the average, while the dotted horizontal lines indicate the standard deviation.

FIG. 6 provides a graph showing the proposed relationship between θ_{E} values and taxonomic distance in a taxonomic continuum. As the taxonomic continuum is multi-dimensional, it is also possible to show genetic similarity peaks in multi-dimensional structures. However, this Figure is shown as a two-dimensional graph for convenience. The dashed lines indicate the degree of conservation of genome fragments with different θ_{E} values.

### DESCRIPTION OF THE INVENTION

The present invention provides methods and compositions for the identification of microbial species from a variety of sources, including clinical specimens, food, environmental samples, waste or drinking water samples and industrial samples. In particularly preferred embodiments, the present invention provides a DNA “chip” containing bacterial genomic sequences arranged in a microarray for bacterial identification.

Indeed, the present invention provides a new approach to identify and type bacteria based on genomic DNA-DNA similarity that eliminates the disadvantages of prior art methods. As discussed in greater detail below, in preferred embodiments, the methods provided by the present invention take advantage of the capacity provided by microarray technology. Also as discussed in greater detail below, in these preferred embodiments, bacterial genomes are fragmented randomly and representative fragments are spotted on a glass slide and then hybridized to test genomes. Resulting hybridization profiles are used in statistical procedures to identify test strains. Importantly, a database of hybridization profiles is established.

In some preferred embodiments, reference sequences are prepared by random cloning of genomic sequences in the 1 to 2 kb range from multiple reference species (strains) and subsequent amplification of the genomic inserts. The purified amplification products are then arrayed by a printing process. Genomic DNA from test strains (i.e., organisms to be characterized) is labeled by random priming, and co-hybridized to the chip with reference DNA, which is a mixture of genomic DNAs from the multiple reference species (strains). The hybridized chips are then laser scanned, and a hybridization ratio (test DNA/reference DNA) for each spot on the array is determined, with a correction made for labeling efficiency. Analysis of the corrected hybridization ratios is performed using a correlation coefficient, which can then be expressed as a similarity coefficient to compare hybridization profiles. Clustering analysis is also used to identify the test strains based on hybridization profiles.

In one particular embodiment, the present invention provides a chip arrayed with genomic fragments from reference genomes of four fluorescent Pseudomonas strains. In one embodiment, 60 to 96 genome fragments of approximately 1 kb from each species were spotted on microarrays. Genomes from 12 well-characterized fluorescent Pseudomonas strains were labeled with Cy dyes and hybridized to the arrays. High reproducibility in repeated experiments was observed, and the similarity coefficients calculated based on hybridization to the chip agreed well with DNA-DNA homology-based relationships as determined by % DNA-DNA reassociation values in other reports. Cluster analysis grouped the test strains in agreement with other types of experimental data (16S rDNA sequence and % DNA-DNA homology). Many species were clearly resolved in the cluster analysis, while some pairs were not resolved. In addition to identification, the analysis was able to characterize the degree of conservation of the sequences on the array.

In addition to these embodiments utilizing Pseudomonas species, other embodiments of the present invention provide larger arrays, such as mixture of DNA from both Gram-negative and Gram-positive species. The present invention also provides methods to create databases of hybridization profiles for comparisons with future test strains. Also, in one embodiment, arrays containing up to approximately 100,000 DNA spots are used. Thus, a single array is capable of providing broad resolution and identification capacity. Furthermore, the present invention overcomes disadvantages associated with traditional DNA-DNA hybridization methods commonly used. For example, laborious cross-hybridizations are avoided and an open database (i.e., a reference database to match chip contents) of hybridization profiles is made possible by the present invention.

In some embodiments, genome fragments from additional reference strains are spotted on the array, as this tends to increase the resolution and the consistency of the approach used in the present invention. In the Examples herein, the use of 338 genome fragments from four reference strains are described. Considering that the average genome size of fluorescent Pseudomonas strains is approximately 5 Mb and that the size of the genome fragments used was 1 to 2 Kb, the array used in these Examples sampled approximately 1 to 3% of a genome. However, assuming that each spot (i.e., genome fragment) tests individual genetic characteristics quantitatively, the array performed 338 individual tests for determining the similarity coefficients per one test strain. Sokal and Sneath (Sokal and Sneath, Principles of Numerical Taxonomy, W. H. Freeman & Co., San Francisco [1963]) suggest that use of more than 60 characters gives significant reliability for similarity coefficients and enough information for numerical taxonomy. In fact, all of similarity coefficients obtained as described in the Examples were statistically significant (P<0.0001).

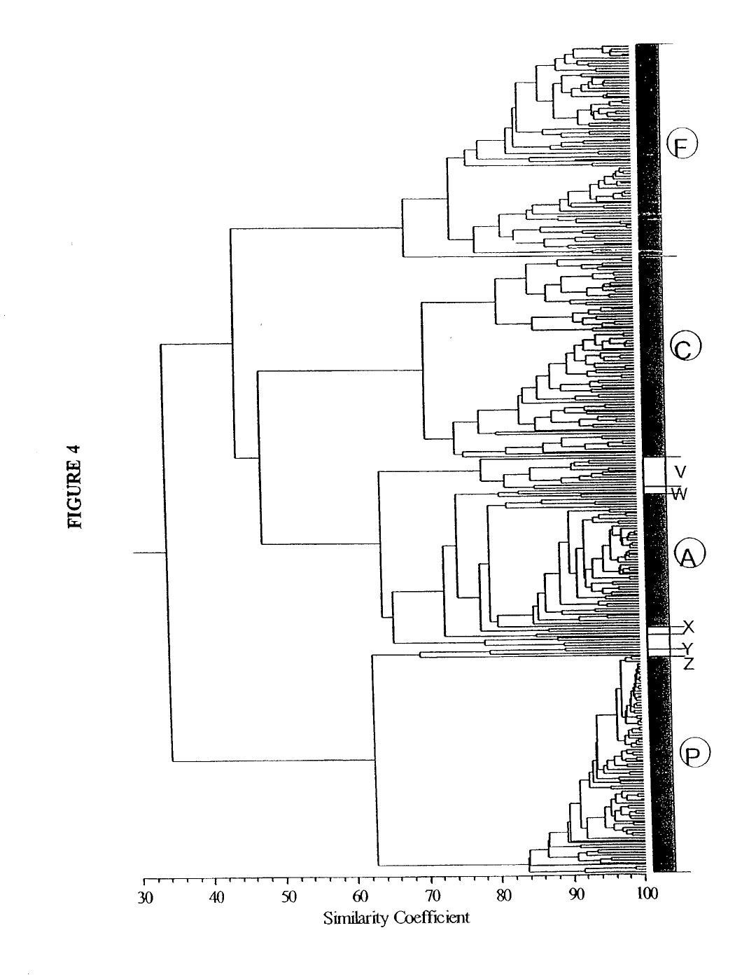

Cluster analysis was also performed on the hybridization patterns of all 338 spotted fragments across all strain tested. As shown in FIG. 4, four main clusters were found at a cophenetic similarity of 70%. Main clusters F, C, A, and P were mainly comprised of the fragments from the four reference strains, P. fluorescens (98.7%), P. chlororaphis (94.1%), P. aeruginosa (91.8%), and P. putida (100%), respectively. Minor clusters V, W, X, and Z were comprised of the genome fragments from different reference strains. In gene expression data analysis, such clusters indicate that these genes tend to turn on and turn off simultaneously, but the grouping in this study indicates only that the hybridization patterns of the cluster member are similar at a certain degree. Formation of a cluster of genome fragments from different reference strains suggests, but does not confirm, conserved sequences.

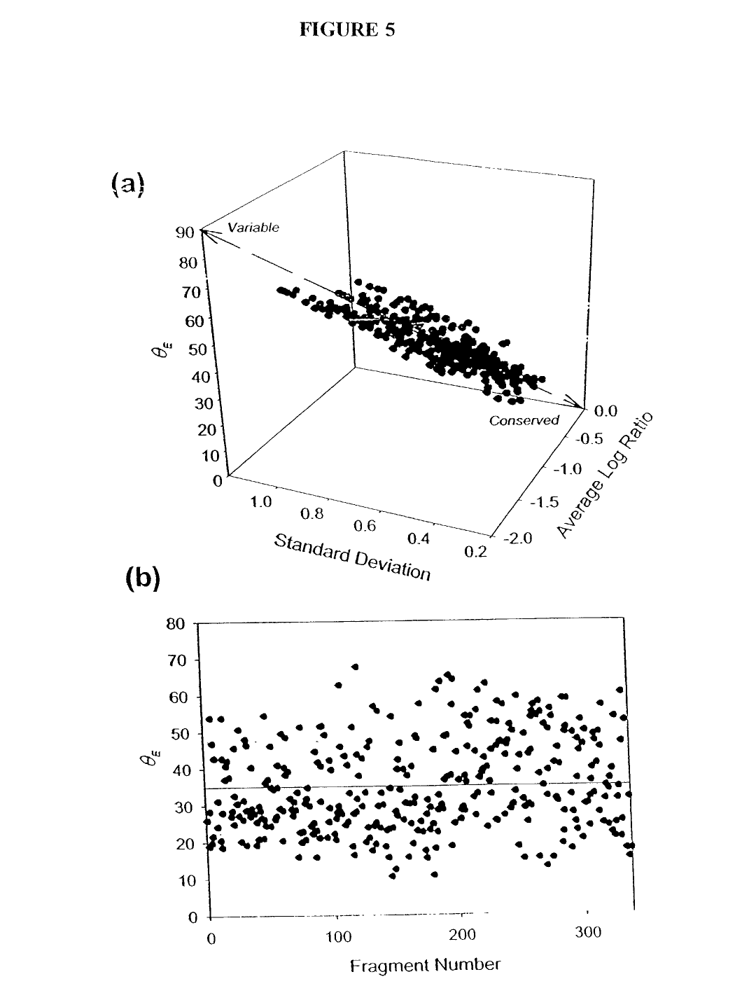

To conveniently find conserved and unique (variable) sequences in the fragment collection described in the Examples, an evenness index (E) (Legendre and Legendre, Numerical Ecology, Elsevier Science, Amsterdam [1998]; Pielou, J. Theor. Biol., 13:131-144 [1966]) was calculated from hybridization signal ratio profiles of each spotted genome fragment across the test strains. These results are shown in FIG. 5. For fragments that are extremely conserved in all test strains (e.g., rRNA genes), the angle (θ_{E}) shows its minimum value (0°). Genomic fragments showing a small angle (high evenness) tend to show a high hybridization signal ratio with low standard deviation, indicating that they have an equally high hybridization signal to many genomes tested. Hence, they can be considered to be conserved sequences. In contrast, genomic fragments with a large angle (low evenness) tend to show a low average signal ratio with high standard deviation, indicating that they have an appreciable hybridization signal only to the closely related strains. Hence, these are considered to be variable sequences.

The average angle (θ_{E}) for all data was 35.0°±12.5°. Fifty one (15.1%) fragments of θ_{E} values lower than one standard deviation (S.D.) below the mean (<22.5°) (See, FIG. 5, Panel B) showed appreciable hybridization signal (R′>1) for the genomic DNAs from closely related species (e.g., species pairs P. fluorescens and P. marginalis; and P. chlororaphis and P. aureofaciens). The majority of these originated from two reference strains P. fluorescens (ATCC 13525^{T}) and P. chlororaphis (ATCC 9447), including only five fragments from the clusters V, W, X, and Z, as shown in FIG. 4. Fragments showing appreciable hybridization (R′>1) for all strains tested (e.g., θ_{E}<<10°), were not found on the array obtained as described in the present Examples.

Sixty eight (20.1%) fragments with θ_{E} values one S.D. above the mean (>47.5°) showed appreciable hybridization only when hybridized to the reference strains. The rest of the fragments (64.8%) showed an intermediate level of conservation. While four main clusters (F, P, C, and A; as shown in FIG. 4) contain all genome fragments with θ_{E} values of 22.5° to 47.5° (at the species level), the groups of highly variable sequences (θ_{E} value>47.5°) (at the strain level) are also located in the main clusters (See, FIG. 4). It is noteworthy that the variable and conserved sequences cannot be reliably identified by cluster analysis (See, FIG. 4), but are easily revealed by θ_{E} values.

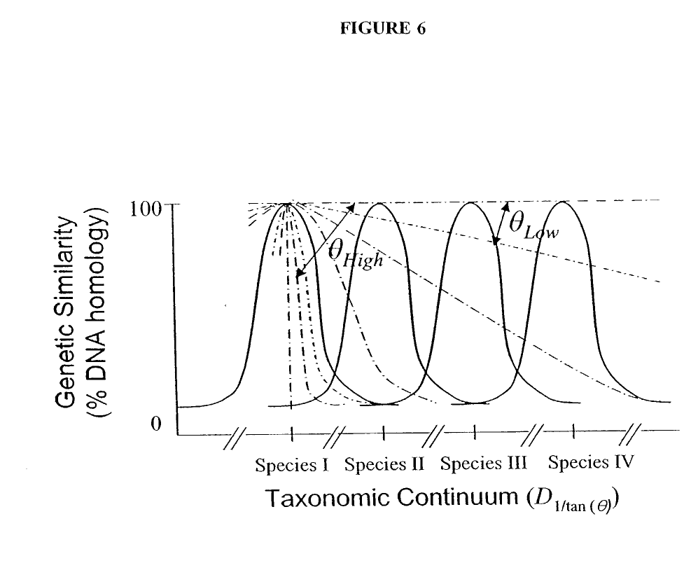

The calculated θ_{E} values are also useful for constructing relationships between θ_{E} values and taxonomic distance (See, FIG. 6), where valley-shaped regions are considered to be caused by selection pressure, resulting in subsequent speciation events. The genome fragments with low θ_{E} values have almost identical sequences, and are distributed over a wide taxonomic range, while the fragments with high θ_{E} values are distributed over a narrow taxonomic range. When the empirical results obtained in the Examples described herein (i.e., θ_{E} values) were applied to this diagram, the degree of conservation within strain level, species level, closely related species level, and genus level roughly corresponded to θ_{E} values of >50°, 50° to 20°, 20° to 10°, and <10°, respectively. Additionally, a taxonomic distance (D_{1/tan(θ)}) was calculated (D_{1/tan(θ)}=1/[tan(θ_{E})]). The range of θ_{E} values for species level (>20°) in the experiments described in the Examples resulted in a D_{1/tan(θ)} of 2.74, indicating a radius of taxonomic range for a species. This alternative to calculating taxonomic distance using genome-wide analyses finds use in delineating species, although the values would be expected to vary with the microbial group tested.

Thus, the present invention provides methods and compositions for the identification of microorganisms (e.g., bacteria) using DNA-DNA hybridization with DNA microarrays. Although the present Examples involved testing on four reference strains of fluorescent Pseudomonas, it is not intended that the present invention be limited to this genus. Indeed, the methods of the present invention are suitable for use with various microorganisms. Given the current technology of microarray fabrication, it is possible to spot 100,000 genomic fragments on a chip. Hence, it is feasible to test 1000 reference strains with 100 genome fragments from each reference strain. Although arrays of this size are sufficient to cover the full taxonomic range of either gram-negative or gram-positive bacteria, smaller or larger arrays are provided by the present invention. In addition, combinations of arrays find use in the extended analysis and comparisons of organisms. The methods of the present invention find use in determining the genetic distances among microorganisms, as well as for identifying species of microorganisms. In particularly preferred embodiments, the methods are used for the analysis of bacteria. However, it is not intended that the present invention be limited to bacteria, as the present invention finds use with other microorganisms as well.

The present invention provides major improvements and advantages over the traditional DNA-DNA reassociation approaches commonly used. For example, the present invention does not need cross hybridization to identify genetic relationships between test strains, does not require the use of an isotope, and is capable of utilizing an open database of hybridization profiles when standard genome chips for bacteria are available. Indeed, unlike other methods presently available, the present invention permits the capture of hybridization information from any microbial species. The use of multiple probes and multiple reference species, that can be customized for the user's purposes, as needed, provides means for great capability in identification and characterization of microorganisms. For example, although the prototype described herein utilized only approximately 380 genome fragments from four different Pseudomonas species as reference strains, in other embodiments, the arrays of the present invention are constructed with probes from at least 5000 different species. Thus, using the method of the present invention, only 5×10⁵ probes are needed to cover the majority of bacterial species.

Definitions

To facilitate an understanding of the present invention, a number of terms and phrases are defined below:

As used herein, the terms “microbe” and “microbial” refer to microorganisms. In particularly preferred embodiments, the microbes identified using the present invention are bacteria (i.e., eubacteria and archaea). However, it is not intended that the present invention be limited to bacteria, as other microorganisms are also encompassed within this definition, including fungi, viruses, and parasites (e.g., protozoans and helminths).

As used herein, the term “reference DNA” refers to DNA that is obtained from a known organism (i.e., a reference strain). In some embodiments of the invention, the reference DNA comprises random genome fragments. In particularly preferred embodiments, the genome fragments are of approximately 1 to 2 kb in size. Thus, in preferred embodiments, the reference DNA of the present invention comprises mixtures of genomes from multiple reference strains.

As used herein, the term “multiple reference strains” refers to the use of more than one reference strains in an analysis. In some embodiments, multiple reference strains within the same species are used, while in other embodiments, “multiple reference strains” refers to the use of multiple species within the same genus, and in still further embodiments, the term refers to the use of multiple species within different genera.

As used herein, the terms “test DNA” and “sample DNA” refer to the DNA to be analyzed using the method of the present invention. In preferred embodiments, this test DNA is tested in the competitive hybridization methods of the present invention, in which reference DNA(s) from multiple reference strains is/are used.

As used herein, the term “reference strain” and “reference species” refer to microorganisms with known characteristics. In some cases reference strains are recognized as “type cultures” or “standard strains.”

As used herein, the term “taxonomy” refers to the science of identification, classification, and nomenclature of organisms.

As used herein, the term “evolutionary distance” refers to the sum of the physical distance in a phylogenetic tree that separates organisms; this distance is inversely proportional to evolutionary relatedness.

As used herein, the term “phylogeny” refers to the evolutionary history of organisms.

As used herein, the term “signature sequence” refers to short oligonucleotides of defined sequence in 16S or 18S rRNA, that are characteristic of specific organisms or a group of related organisms.

As used herein, the term “genus” refers to organisms within a particular tribe (or subtribe), that share genotypic and phenotypic characteristics that are different from other members of the tribe (or subtribe). A genus is usually a collection of different species, each sharing one or more major properties.

As used herein, the term “species” refers to organisms within a particular genus (e.g., Pseudomonas aeruginosa is within the genus Pseudomonas). Isolates of organisms within the same species share genotypic, as well as phenotypic characteristics. Within species, there are “groups” and “types,” and “strains” that share genotypic and phenotypic characteristics. Indeed, a species can be described as collection of strains which all share the same major properties, but which differ in one or more significant properties from other collections of strains.

As used herein, the terms “numeral taxonomy” and “numerical taxonomy” refer to the use of a large number of characters (e.g., phenotypic characteristics) given equal weight in grouping strains. The “similarity coefficient” for two strains is the number of positive phenotypic characteristics that they share divided by the total number of positive characteristics in either strain or both strains.

As used herein, the term “molecular taxonomy” refers to the use of molecular methods to determine the relatedness between organisms. Molecular taxonomy is based on the use of DNA or protein sequences to measure the evolutionary relatedness between species. In most methods, the differences between organisms are measured in terms of DNA composition, sequence homology (e.g., as assessed by hybridization of DNA and/or RNA), and protein sequences.

As used herein, the term “genotype” refers to the entire genetic constitution of an organism, while the term “phenotype” refers to the entire physical, biochemical, and physiological makeup of an organism (e.g., the readily observable characteristics), as determined both genetically and by the environment.

The terms “sample” and “specimen” in the present specification and claims are used in their broadest sense. On the one hand, they are meant to include a specimen or culture. On the other hand, they are meant to include both biological and environmental samples. These terms encompasses all types of samples obtained from humans and other animals, including but not limited to, body fluids such as urine, blood, fecal matter, cerebrospinal fluid (CSF), semen, and saliva, as well as solid tissue. These terms also refers to swabs and other sampling devices which are commonly used to obtain samples for culture of microorganisms.

Biological samples may be animal, including human, fluid or tissue, food products and ingredients such as dairy items, vegetables, meat and meat by-products, and waste. Environmental samples include environmental material such as surface matter, soil, water, and industrial samples, as well as samples obtained from food and dairy processing instruments, apparatus, equipment, disposable, and non-disposable items. These examples are not to be construed as limiting the sample types applicable to the present invention.

Whether biological or environmental, a sample suspected of containing microorganisms may (or may not) first be subjected to an enrichment means to create a “pure culture” of microorganisms. By “enrichment means” or “enrichment treatment,” the present invention contemplates (i) conventional techniques for isolating a particular microorganism of interest away from other microorganisms by means of liquid, solid, semi-solid or any other culture medium and/or technique, and (ii) novel techniques for isolating particular microorganisms away from other microorganisms. It is not intended that the present invention be limited only to one enrichment step or type of enrichment means. For example, it is within the scope of the present invention, following subjecting a sample to a conventional enrichment means, to subject the resultant preparation to further purification such that a pure culture of a strain of a species of interest is produced. This pure culture may then be analyzed by the methods of the present invention.

As used herein, the term “primary isolation” refers to the process of culturing organisms directly from a sample. Thus, primary isolation involves such processes as inoculating an agar plate from a culture swab, urine sample, environmental sample, etc. Primary isolation may be accomplished using solid or semi-solid agar media, or in liquid. As used herein, the term “isolation” refers to any cultivation of organisms, whether it be primary isolation or any subsequent cultivation, including “passage” or “transfer” of stock cultures of organisms for maintenance and/or use.

As used herein, the term “culture” refers to any sample or specimen which is suspected of containing one or more microorganisms or cells. In particularly preferred embodiments, the term is used in reference to bacteria and fungi. “Pure cultures” are cultures in which the organisms present are only of one strain of a particular genus and species. This is in contrast to “mixed cultures,” which are cultures in which more than one genus and/or species of microorganism are present.

As used herein, the terms “microbiological media” and “microbiological culture media,” and “media” refer to any substrate for the growth and reproduction of microorganisms. “Media” may be used in reference to solid plated media which support the growth of microorganisms. Also included within this definition are semi-solid and liquid microbial growth systems including those that incorporate living host organisms, as well as any type of media.

As used herein, the terms “culture media,” and “cell culture media,” refers to media that are suitable to support the growth of cells in vitro (i.e., cell cultures). It is not intended that the term be limited to any particular cell culture medium. For example, it is intended that the definition encompass outgrowth as well as maintenance media. Indeed, it is intended that the term encompass any culture medium suitable for the growth of the cell cultures of interest.

As used herein, the term “cell type,” refers to any cell, regardless of its source or characteristics.

As used herein, the term “cell line,” refers to cells that are cultured in vitro, including primary cell lines, finite cell lines, continuous cell lines, and transformed cell lines.

As used herein, “light beam” refers to directed light, for example, comprised of either a continuous cross-section or a plurality of convergent or divergent sub-beams (e.g., a patterned beam). This term is meant to include, but is not limited to, light emitted from a light source, laser, light reflected upon striking a reflecting device (e.g., a micromirror), and the like.

As used herein, “optical signal” refers to any energy (e.g., photodetectable energy) from a sample (e.g., produced from a microarray that has one or more optically excited [i.e., by electromagnetic radiation] molecules bound to its surface).

As used herein, “filter” refers to a device or coating that preferentially allows light of a characteristic spectra to pass through it (e.g., the selective transmission of light beams).

As used herein, the term “spatial light modulator” refers to devices that are capable of producing controllable (e.g., programmable by a processor, or pre-directed by a user), spatial patterns of light from a light source. Spatial light modulators include, but are not limited to, Digital Micromirror Devices (DMDs) and Liquid Crystal Devices (LCDs). In some embodiments, the LCD is reflective, while in other embodiments, it is transmissive of the directed (e.g., spatially modulated) light.

“Polychromatic” and “broadband” as used herein, refer to a plurality of electromagnetic wavelengths emitted from a light source.

As used herein, “microarray” refers to a substrate with a plurality of molecules (e.g., nucleotides) bound to its surface. Microarrays, for example, are described generally in Schena, Microarray Biochip Technology, Eaton Publishing, Natick, Mass., (2000). Additionally, the term “patterned microarrays” refers to microarray substrates with a plurality of molecules non-randomly bound to its surface.

As used herein, the term “micromirror array” refers to a plurality of individual light reflecting surfaces that are addressable (e.g., electronically addressable in any combination), such that one or more individual micromirrors can be selectively tilted, as desired.

As used herein, the terms “optical detector” and “photodetector” refers to a device that generates an output signal when exposed to optical energy. Thus, in its broadest sense, the term “optical detector system” refers devices for converting energy from one form to another for the purpose of measurement of a physical quantity and/or for information transfer. Optical detectors include but are not limited to photomultipliers and photodiodes, as well as fluorescence detectors.

As used herein, the term “TTL” stands for Transistor-Transistor Logic, a family of digital logic chips that comprise gates, flip/flops, counters etc. The family uses zero Volt and five Volt signals to represent logical “0” and “1” respectively.

As used herein, the term “dynamic range” refers to the range of input energy over which a detector and data acquisition system is useful. This range encompasses the lowest level signal that is distinguishable from noise to the highest level that can be detected without distortion or saturation.

As used herein, the term “noise” in its broadest sense refers to any undesired disturbances (i.e., signal not directly resulting from the intended detected event) within the frequency band of interest. Noise is the summation of unwanted or disturbing energy introduced into a system from man-made and natural sources. Noise may distort a signal such that the information carried by the signal becomes degraded or less reliable.

As used herein, the term “signal-to-noise ratio” refers the ability to resolve true signal from the noise of a system. Signal-to-noise ratio is computed by taking the ratio of levels of the desired signal to the level of noise present with the signal. In preferred embodiments of the present invention, phenomena affecting signal-to-noise ratio include, but are not limited to, detector noise, system noise, and background artifacts. As used herein, the term “detector noise” refers to undesired disturbances (i.e., signal not directly resulting from the intended detected energy) that originate within the detector. Detector noise includes dark current noise and shot noise. Dark current noise in an optical detector system results from the various thermal emissions from the photodetector. Shot noise in an optical system is the product of the fundamental particle nature (i.e., Poisson-distributed energy fluctuations) of incident photons as they pass through the photodetector.

As used herein, the term “system noise” refers to undesired disturbances that originate within the system. System noise includes, but is not limited to noise contributions from signal amplifiers, electromagnetic noise that is inadvertently coupled into the signal path, and fluctuations in the power applied to certain components (e.g., a light source)

As used herein, the term “background artifacts” include signal components caused by undesired optical emissions from the microarray. These artifacts arise from a number of sources, including: non-specific hybridization, intrinsic fluorescence of the substrate and/or reagents, incompletely attenuated fluorescent excitation light, and stray ambient light. In some embodiments, the noise of an optical detector system is determined by measuring the noise of the background region and noise of the signal from the microarray feature.

As used herein, the term “processor” refers to a device that performs a set of steps according to a program (e.g., a digital computer). Processors, for example, include Central Processing Units (“CPUs”), electronic devices, and systems for receiving, transmitting, storing and/or manipulating digital data under programmed control.

As used herein, the terms “memory device,” and “computer memory” refer to any data storage device that is readable by a computer, including, but not limited to, random access memory, hard disks, magnetic (e.g., floppy) disks, zip disks, compact discs, DVDs, magnetic tape, and the like.

The term “gene” refers to a nucleic acid (e.g., DNA) sequence that comprises coding sequences necessary for the production of a polypeptide or precursor. It is intended that the term encompass polypeptides encoded by a full length coding sequence, as well as any portion of the coding sequence, so long as the desired activity and/or functional properties (e.g., enzymatic activity, ligand binding, etc.) of the full-length or fragmented polypeptide are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as “5′ untranslated sequences.” The sequences that are located 3′ (i.e., “downstream”) of the coding region and that are present on the mRNA are referred to as “3′ untranslated sequences.” The term “gene” encompasses both cDNA and genomic forms of a gene. A genomic form of a genetic clone contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” and “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” and “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification and hybridization reactions, as well as detection methods that depend upon binding between nucleic acids.

The terms “homology” and “similarity” refer to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The inhibition of hybridization of the completely complementary sequence to the target sequence may be examined using a hybridization assay (e.g., Southern and/or Northern blots, solution hybridization and the like) under conditions of low stringency. A substantially homologous sequence or probe competes for and inhibits the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides they may be single-stranded or double-stranded. Probes are useful in the detection, identification and isolation of particular targets (e.g., gene sequences). In some embodiments, it is contemplated that probes used in the present invention are labelled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular label. With respect to microarrays, the term probe is used to refer to any hybridizable material that is affixed to the microarray for the purpose of detecting “target” sequences in the analyte.

As used herein “probe element” and “probe site” refer to a plurality of probe molecules (e.g., identical probe molecules) affixed to a microarray substrate. Probe elements containing different characteristic molecules are typically arranged in a two-dimensional array, for example, by microfluidic spotting techniques or by patterned photolithographic synthesis, etc.

As used herein, the term “target,” when used in reference to hybridization assays, refers to the molecules (e.g., nucleic acid) to be detected. Thus, the “target” is sought to be sorted out from other molecules (e.g., nucleic acid sequences) or is to be identified as being present in a sample through its specific interaction (e.g., hybridization) with another agent (e.g., a probe oligonucleotide). A “segment” is defined as a region of nucleic acid within the target sequence.

As used herein, the term “polymerase chain reaction” (“PCR”) refers to the methods described in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications. With PCR, it is possible to amplify a single copy of a specific target sequence in genomic DNA to a level detectable by the device and systems of the present invention.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “reverse-transcriptase” and “RT-PCR” refer to a type of PCR where the starting material is mRNA. The starting mRNA is enzymatically converted to complementary DNA or “cDNA” using a reverse transcriptase enzyme. The cDNA is then used as a “template” for a “PCR” reaction.

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell genome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

The term “sequences associated with a genome” means preparations of genomes (e.g., spreads of metaphase chromosomes of eukaryotes), nucleic acid extracted from a sample containing DNA (e.g., preparations of genomic DNA); the RNA that is produced by transcription of genes located on a chromosome (e.g., hnRNA and mRNA); and cDNA copies of the RNA transcribed from the DNA located in a genome. Sequences associated with a genome may be detected by numerous techniques including probing of Southern and Northern blots and in situ hybridization to RNA, DNA (or metaphase chromosomes) with probes containing sequences homologous to the nucleic acids in the above listed preparations.

As used herein the term “coding region” when used in reference to a structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets which specify stop codons (i.e., TA, TAG, TGA).

As used herein, the terms “purified” and “to purify” refer to the removal of contaminants from a sample.

The term “recombinant DNA molecule” as used herein refers to a DNA molecule that is comprised of segments of DNA joined together by means of molecular biological techniques.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide.

The terms “recombinant protein” and “recombinant polypeptide” as used herein refer to a protein molecule that are expressed from a recombinant DNA molecule.

As used herein the term “biologically active polypeptide” refers to any polypeptide which maintains a desired biological activity.

As used herein the term “portion” when in reference to a protein (as in “a portion of a given protein”) refers to fragments of that protein. The fragments may range in size from four amino acid residues to the entire amino acid sequence minus one amino acid.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); BSA (bovine serum albumin); CFA (complete Freund's adjuvant); IFA (incomplete Freund's adjuvant); IgG (immunoglobulin G); IM (intramuscular); IP (intraperitoneal); IV (intravenous or intravascular); SC (subcutaneous); H₂O (water); HCl (hydrochloric acid); aa (amino acid); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); PMSF (phenylmethylsulfonyl fluoride); RT-PCR (reverse transcription PCR); SDS (sodium dodecyl sulfate); Tris (tris(hydroxymethyl)aminomethane); SSC (3 M NaCl, 0.3 M trisodium citrate 2 H₂O, pH 7.0); w/v (weight to volume); v/v (volume to volume); Amersham (Amersham Pharmacia, Piscataway, N.J.); ICN (ICN Pharmaceuticals, Inc., Costa Mesa, Calif.); Amicon (Amicon, Inc., Beverly, Mass.); ATCC (American Type Culture Collection, Manassus, Va.); Becton Dickinson (Becton Dickinson Labware, Lincoln Park, N.J.); BioRad (BioRad, Richmond, Calif.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); New England Biolabs (New England Biolabs, Inc., Beverly, Mass.); Novagen (Novagen, Inc., Madison, Wis.); Pharmacia (Pharmacia, Inc., Piscataway, N.J.); Sigma (Sigma Chemical Co., St. Louis, Mo.); Stratagene (Stratagene, La Jolla, Calif.); Coming (Coming Co., Coming, N.Y.); Molecular Probes (Molecular Probes, Eugene, Oreg.); Qiagen (Qiagen, Valencia, Calif.); Roche (Roche, Indianapolis, Ind.); Axon (Axon, Foster City, Calif.); SPSS (SPSS, Chicago, Ill.); and Exeter (Exeter Software, East Setauket, N.Y.).

In these experiments, Pseudomonas fluorescens (ATCC 13525^{T}), P. fluorescens (ATCC 17397), P. fluorescens (ATCC 17400), P. fluorescens (ATCC 17467), P. fluorescens (ATCC 33512), P. marginalis (LMG 5039), P. chlororaphis (ATCC 9447), P. chlororaphis (ATCC 17811), P. aureofaciens (ATCC 13985^{T}), P. putida (ATCC 12633^{T}), P. aeruginosa (ATCC 15692), and P. aeruginosa (ATCC 17429) were used. All strains were routinely cultivated at 30° C. in nutrient broth medium (Difco). Genomic DNAs from the strains were extracted and purified using Genomic Tips (Qiagen) with Genomic DNA Buffer Set (Qiagen). The concentration of DNA was determined by UV spectrophotometry and with SpotCheck (Sigma).

### Example 1

### Microarray Fabrication

In this Example, the production of one microarray embodiment is described. Genomic DNAs from four fluorescent Pseudomonas strains (Pseudomonas fluorescens (ATCC 13525^{T}), P. chlororaphis (ATCC 9447), P. putida (ATCC 12633^{T}), and P. aeruginosa (ATCC 15692); i.e., the “reference strains”) were fragmented by bead-beating to ensure randomness, and the fragments were size-fractionated (1 to 2 kb) by agarose gel electrophoresis, as known in the art. The QIAquick Gel Extraction Kit (Qiagen) was used to elute and purify DNA from the agarose gel, according to the manufacturer's instructions. The genomic DNA fragments were inserted to pPCR-Script Amp vector (Stratagene), then PCR amplified with the T3/T7 promoter primer set using standard PCR conditions, with a primer annealing temperature of 55° C. Amplified genomic DNA fragments were purified with QIAquick 8 PCR purification kit (Qiagen) and quantified with PicoGreen (Molecular Probes), according to the manufacturer's instructions.

Purified DNAs were resuspended (200 ng/μl) in 3×SSC (1×SSC is 0.15 M NaCl, plus 0.015 M sodium citrate), and printed using approximately 1 nl/spot, on CMT-GAPS amino silane coated slides (Coming). In these experiments, 92, 90, 96, and 60 fragments from P. fluorescens, P. chlororaphis, P. putida, and P. aeruginosa were spotted in duplicate, respectively. Yeast gene STE (pheromone receptor gene; GenBank accession no. M12239) was spotted as positive control, and yeast gene ACT (actin gene; GenBank accession no. L00026), lambda DNA, and water were spotted as negative controls. PCR primer pair, STE3F1 (CCC CTT CAA AAT TGG AGC TTG C; SEQ ID NO: 1) and STE3R1 (CCC CCT TTA GCA TGG CAT TCA; SEQ ID NO: 2), and pair ACT1F1 (GAT GGA GCC AAA GCG GTG A (SEQ ID NO: 3) and ACT1R1 (GCG CTT GCA CCA TCC CAT T; SEQ ID NO: 4) were used to amplify the STE and ACT yeast genes, respectively.

After drying, the slides were processed with the succinic anhydride blocking method according to the manufacturer's protocol and stored at room temperature until used.

### Example 2

### Genomic DNA Labeling and Hybridization

In this Example, labeling and hybridization experiments conducted using one embodiment of the present invention are described. Genomic DNAs (1 μg) from all the strains listed above, including the reference strains, were labeled with FluoroLink Cy3-dCTP (Amersham) by random priming using High Prime (Roche), and used as test DNAs. Mixtures of genomic DNA (1 μg) from the four reference strains (1:1:1:1) used for microarray fabrication were labeled with FluoroLink Cy5-dCTP (Amersham) and used as reference DNA for signal ratio calculation (Cy3-Test/Cy5-Ref). Yeast gene STE (10 ng) was included in each labeling reaction as a positive control, as well as an internal standard (IS; Cy3-IS and Cy5-IS) for labeling efficiency correction.

The arrays were pre-hybridized in pre-hybridization buffer (3.5×SSC, 0.1% SDS, 10 mg/ml bovine serum albumin) for 20 min at 65° C., hybridized with approximately 1 μg of Cy3- and Cy5-labeled DNA mixture (1:1) in hybridization buffer (3×SSC, 0.1% SDS, 0.5 mg/ml yeast tRNA) at 65° C. overnight, then washed once with primary wash buffer (0.1×SSC, 0.1% SDS) at room temperature for 5 min and twice with secondary wash buffer (0.1×SSC) for 5 min.

### Example 3

### Statistical Analyses of Hybridized Arrays

In this Example, the methods used to analyze the data obtained from the hybridized arrays of one embodiment of the present invention are described. Hybridized arrays were scanned with a GenePix 4000 laser scanner (Axon). Laser lights of wavelength at 532 and 635 nm were used to excite Cy3 dye and Cy5 dyes, respectively. Fluorescent images were captured as multi-image-tagged image file format (TIFF) and analyzed with GenePix Pro 3.0 software (Axon). The ratio (R) of the extent of hybridization between test DNAs and reference DNAs was derived from a median value of pixel-by-pixel ratios. By using this approach to calculate R, non-specific signals, which appear in both wavelength images, were found to have less of an effect than when the mean values of a whole spot were used.

Hybridization signal ratios (R) between test DNA and reference DNA (Cy3-Test/Cy5 Test/Cy5-Ref) were calculated and corrected with the correction factor (c=Cy5-IS/Cy3-IS) from the internal standard (yeast gene STE) (corrected signal ratio R′=c×[Cy3-Test/Cy5-Ref]). Spearman correlation coefficients (r) were calculated to find relationships between hybridization patterns and transformed to a percentage scale. Unweighted arithmetic average clustering (UPGMA) was used for hierarchical data ordination. For characterizing the shape of hybridization signal distribution, an evenness (E) value of each spotted genome fragment was calculated based on information theory (Legendre and Legendre, Numerical Ecology, Elsevier Science, Amsterdam [1998]; and Pielou, J. Theor. Biol., 13:131-144 [1966]) using the equation E=(−Σ p log p )/log q; where p is the relative proportion of hybridization signal ratio (R′), and q is the total number of hybridizations performed. Since the distribution of the calculated E values was highly skewed (skewness =−0.855), the E values were normalized using arc cosine transformation. An arc cosine-transformed evenness value, θ_{E}, was used to represent the degree of conservation of each genome fragment. Microsoft EXCEL, SYSTAT (SPSS) and NTSYS-pc (Exeter) were used for all statistical calculations.

The ratio of Cy5 to Cy3 incorporation (Cy5-IS/Cy3-IS) during the DNA labeling was found to be 1.04±0.32 for all experiments. As indicated above, an incorporation ratio (c=Cy5-IS/Cy3-IS) obtained from each microarray was used as a correction factor for hybridization signal calibration (corrected signal ratio R′=c×[Cy3-Test/Cy5-Ref]). The correction factor, however, did not affect the correlation coefficient calculation, since the correlation coefficient is independent of any constant (e.g., “c”).

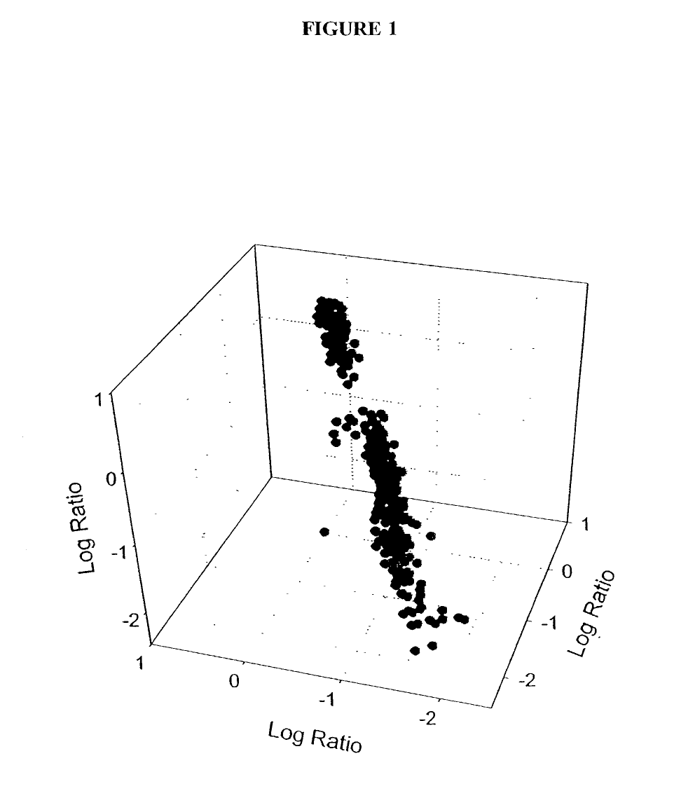

In order to test the reproducibility of array hybridization, seven arrays were hybridized to genomic DNAs of P. fluorescens (ATCC 13525^{T}) (3 times), P. putida (ATCC 12633^{T}) (2 times), and P. aeruginosa (ATCC 15692) (2 times). FIG. 1 shows the scatter plot representation of triplicate hybridization profiles of Pseudomonas fluorescens (ATCC 13525^{T}). The arrays hybridized to P. fluorescens (ATCC 13525^{T}) (triplicate), P. putida ATCC (12633^{T}) (duplicate), and P. aeruginosa (ATCC 15692) (duplicate) showed similarity values >97.5% (r>0.949, P<0.0001), 95.3% (r=0.906, P<0.0001), and 94.1% (r=0.882, P<0.0001), respectively.

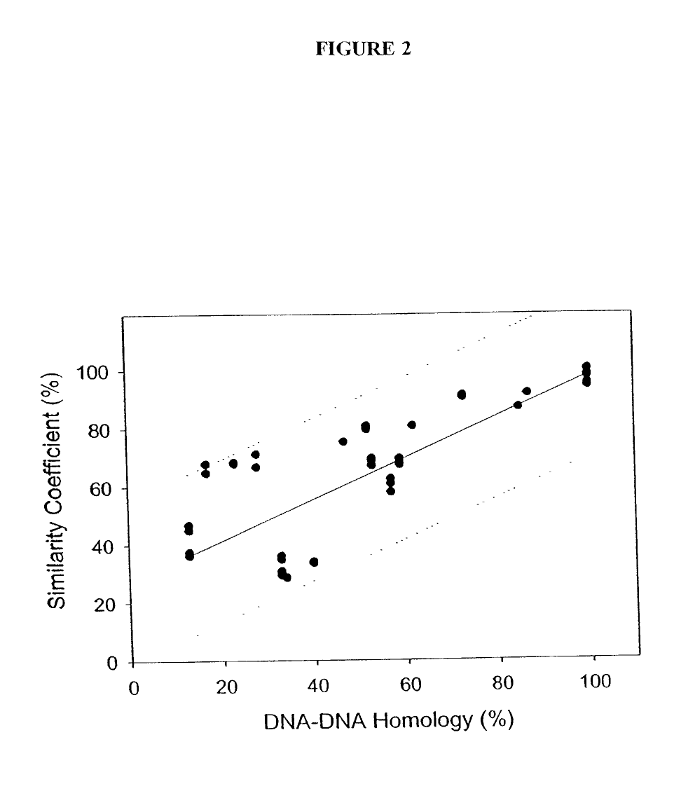

Regression analysis showed a good agreement between DNA-DNA reassociation values and the similarity coefficients obtained from these experiments, as indicated in FIG. 2. For these experiments, the coefficient of determination (r²) was 0.713. Order 1 of linear relationship and the regression coefficient (slope, 0.718) indicated that the microarray method has a similar resolution to the whole genomic DNA-DNA hybridization method. However, the two methods lost their linear relationships below 50% of DNA-DNA similarity, which approximately corresponds to a 60% similarity coefficient obtained by the DNA microarray method.

A similar result was observed with the relationship between repetitive extragenic palindromic (REP)-PCR genomic DNA fingerprint similarity and percent DNA similarity values (Rademaker et al., Int. J. Syst. Evol. Microbiol., 50:665-677 [2000]). REP-PCR fingerprinting (Rademaker et al., in Akkermans et al., Molecular Microbial Ecology Manual, Suppl. 3, Kluwer Academic Publishers, Dordrecht, The Netherlands [1998], pp. 1-26) lost resolution when applied to strains of below 70% of DNA similarity, indicating that REP-PCR fingerprinting is only capable of resolving relationships among very closely related strains.

In contrast, in these experiments, the DNA chip method of the present invention showed linearity over a broader span of DNA similarity values (50 to 100%) but provided slightly less resolution at >70% DNA similarity values than for the REP-PCR fingerprinting method. However, the microarray method of the present invention is still able to resolve closely related strains and, more importantly, provides resolution over the gap between REP-PCR fingerprinting and 16S rDNA analysis (Cho and Tiedje, Abstracts of the 100th General Meeting of the American Society for Microbiology, Abstr. N-171, American Society for Microbiology, Washington, D.C. [2000], at pages 489-490).

In addition, situations in which different strains of the same species have differences in genome size (e.g., E. coli K12, as compared to E. coli 0157; GenBank accession nos. U00096 and AE005174, respectively) were taken into consideration. It is not contemplated that this scale of difference (1 of 5 Mb) will invalidate the methods of the present invention, although the percent similarity should be slightly higher than the average percent similarity from whole-genome DNA-DNA hybridization.

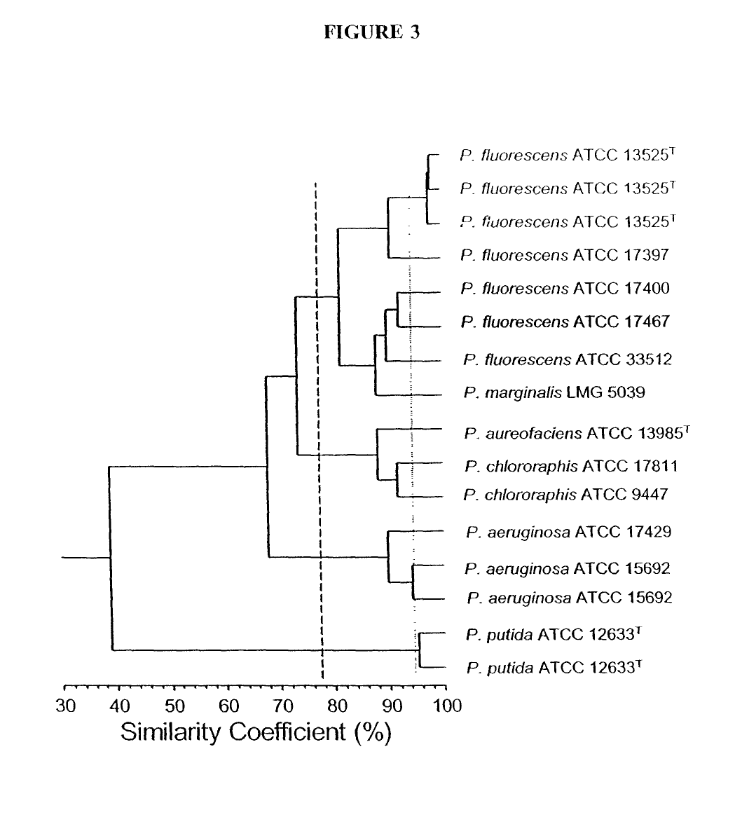

Based on cluster analysis of the overall topology of the dendrogram of similarity coefficient matrix was consistent with the phylogenetic tree obtained from 16S rDNA sequence data (Moore et al., Sys. Appl. Microbiol., 19:478-492 [1996]) except for P. putida and P. aeruginosa clusters, as shown in FIG. 3. The P. aeruginosa group clustered with P. fluorescens and P. chlororaphis groups at a higher similarity (67.9%) than for the P. putida group (39.0%), the latter of which generally shows greater 16S rDNA similarity to P. fluorescens and P. chlororaphis than to P. aeruginosa (Moore et al., supra). However, a similar result to these array data was reported by Palleroni et al. (Palleroni et al., J. Bacteriol., 110:1-11 [1972]), using DNA-DNA similarity values, where the P. aeruginosa group was found to be a closer relative to the P. fluorescens group than was the P. putida group.

All replicate experiments showed similarity coefficients of ≧94% (r=0.88), and all different strains were distinguished at similarity values of ≦91% (r=0.82). Hence, similarity coefficients of <92 to 94% (r=0.84 to 0.88) reliably define different hybridization groups. Using the regression equation from FIG. 1, a cut-off value of 77% was calculated to correspond to a 70% DNA homology value to define “species” (Wayne et al., Int. J. Sys. Bacteriol., 37:463-464 [1987]). This cut-off resolved the P. fluorescens, P. chlororaphis, P. aeruginosa, and P. putida species, but did not resolve P. marginalis from P. fluorescens, nor P. aureofaciens from P. chlororaphis. However, these latter pairs of species are known to be very similar, based on other methods of analysis. For example, P. aureofaciens (ATCC 13985^{T}) and P. chlororaphis (ATCC 9447) show 85% DNA homology (Palleroni et al., supra). In addition, the 16S rDNA similarity between P. aureofaciens and P. chlororaphis, and between P. fluorescens and P. marginalis are 99.5% and 99.9%, respectively (results from different strains) (Moore et al., supra). P. marginalis is also reported to have very similar characteristics to P. fluorescens, and was previously classified as P. fluorescens (Misaghi and Grogan, Phytopathol., 59:1436-1450 [1969]; Pecknold and Grogan, Int. J. Sys. Bacteriol., 23:111-121 [1973]; Sands et al., J. Bacteriol., 101:9-23 [1970]; and Stanier et al., J. Gen. Microbiol., 43:159-271 [1966]). Thus, the present invention provides a means for determining reliable guideline values for species and/or genomovar resolution.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described modes for carrying out the invention which are obvious to those skilled in medicine, diagnostics, evolutionary biology, molecular biology or related fields are intended to be within the scope of the present invention and the following Claims.

We claim:

1. A method of identifying bacteria, comprising:
a) providing:
i) genomic sequences from a plurality of bacterial species arrayed on a solid support so as to create a plurality of arrayed elements, and
ii) labeled target DNA from a test bacteria of interest, and
iii) labeled reference DNA from the strains of bacteria represented on said solid support;
b) hybridizing said target and reference DNA to said arrayed sequences to produce a hybridization pattern, wherein each hybridized DNA in said hybridization pattern has a signal;
c) calculating the ratio of each hybridization signal intensity at each array element to determine the identity of said test bacteria.

2. The method of claim 1, wherein said test bacteria are from a sample obtained from a subject.

3. The method of claim 1, wherein said test bacteria are pathogenic organisms.

4. The method of claim 1, wherein said test bacteria are environmental isolates.

5. The method of claim 1, wherein said solid support is a microchip.

6. The method of claim 1, wherein said calculating comprises statistical analysis.

7. The method of claim 1, wherein said signal comprises fluorescence.

8. The method of claim 1, further comprising the step of producing hybridization profiles of said test and reference bacteria.

9. A method of identifying bacteria, comprising:
a) providing:
i) genomic sequences from a plurality of bacterial species arrayed on at least one microchip, so as to create a plurality of arrayed elements, and
ii) labeled target DNA from a test bacteria of interest, and
iii) labeled reference DNA from the strains of bacteria represented on said at least one microchip;
b) hybridizing said target and reference DNA to said arrayed sequences to produce a hybridization pattern, wherein each hybridized DNA in said hybridization pattern has a signal;
c) calculating the ratio of each hybridization signal intensity at each array element to determine the identity of said test bacteria.

10. The method of claim 9, wherein said test bacteria are from a sample obtained from a subject.

11. The method of claim 10, wherein said test bacteria are pathogenic organisms.

12. The method of claim 9, wherein said test bacteria are environmental isolates.

13. The method of claim 9, further comprising the step of producing hybridization profiles of said test and reference bacteria.

14. The method of claim 9, wherein said calculating comprises statistical analysis.

15. The method of claim 9, wherein said signal comprises fluorescence.

16. A kit for identification of bacteria, comprising genomic sequences from a plurality of bacterial species arrayed on a solid support so as to create a plurality of arrayed elements, and labeled reference DNA from the strains of bacteria represented on said solid support.

17. The kit of claim 16, wherein said solid support comprises at least one microchip.

18. The kit of claim 16, wherein said labeled reference DNA is labeled with a fluorescent label.

19. The kit of claim 16, wherein said reference DNA is obtained from organisms selected from the group consisting of pathogenic bacteria and environmental bacteria.

20. The kit of claim 16, wherein said genomic sequences arrayed on said solid support are labeled.

21. The kit of claim 20, wherein said genomic sequences arrayed on said solid support are labeled with a fluorescent label.
